SEQUENCE LISTING (20 sequences)

SEQ ID NO: 1 - DNA, 19 bp, Artificial
  oligonucleotide being suitable for use as primers for PCR
cctccattgagcggattca

SEQ ID NO: 2 - DNA, 19 bp, Artificial
  oligonucleotide being suitable for use as primers for PCR
gggaatgctggatgcacaa

SEQ ID NO: 3 - DNA, 26 bp, Artificial
  oligonucleotide being suitable for use as primers for PCR
cgcaacgttcaatttaattttgttaa

SEQ ID NO: 4 - DNA, 19 bp, Artificial
  oligonucleotide being suitable for use as primers for PCR
ctgtgaggtcggttgtgcg

SEQ ID NO: 5 - DNA, 14 bp, Artificial
  oligonucleotide being suitable for use as primers for PCR
gccgtgccccggaa

SEQ ID NO: 6 - DNA, 21 bp, Artificial
  oligonucleotide being suitable for use as primers for PCR
catgacccagttcgccatatc

SEQ ID NO: 7 - DNA, 21 bp, Artificial
  oligonucleotide being suitable for use as primers for PCR
tggtctttctgcattcctgga

SEQ ID NO: 8 - DNA, 16 bp, Artificial
  oligonucleotide being suitable for use as primers for PCR
tttggtccacdcgcca

SEQ ID NO: 9 - DNA, 27 bp, Artificial
  oligonucleotide being suitable for use as probes for the real-time PCR
caacactacccggaagcacagttcgtc

SEQ ID NO: 10 - DNA, 25 bp, Artificial
  oligonucleotide being suitable for use as probes for the real-time PCR
cctatggcgtgaaaaccaacgtgca

SEQ ID NO: 11 - DNA, 32 bp, Artificial
  oligonucleotide being suitable for use as probes for the real-time PCR
aatgacgctatgatcccaatctaacttccaca

SEQ ID NO: 12 - DNA, 26 bp, Artificial
  oligonucleotide being suitable for use as probes for the real-time PCR
caactaacgcggcactgtttcccaat

SEQ ID NO: 13 - DNA, 20 bp, Artificial
  universal primer 27F
agagtttgatcmtggctcag

SEQ ID NO: 14 - DNA, 17 bp, Artificial
  universal primer  517R
attaccgcggctgctgg

SEQ ID NO: 15 - DNA, 20 bp, Artificial
  primer vanA1
tctgcaatagagatagccgc

SEQ ID NO: 16 - DNA, 20 bp, Artificial
  primer vanA2
ggagtagctatcccagcatt

SEQ ID NO: 17 - DNA, 20 bp, Artificial
  primer ampC-For
ttctatcaamactggcarcc

SEQ ID NO: 18 - DNA, 20 bp, Artificial
  primers ampC-Rev
ccygttttatgtacccayga

SEQ ID NO: 19 - DNA, 22 bp, Artificial
  primer mecA1
aaaatcgatggtaaaggttggc

SEQ ID NO: 20 - DNA, 22 bp, Artificial
  primer mecA2
agttctgcagtaccggatttgc

(19) United States
(12) Patent Application Publication
(10) Pub. No.: US 2005/0059064 A1
(43) Pub. Date: Mar. 17, 2005

(54) Oligonucleotide, method and system for detecting antibiotic resistance-mediating genes in microorganisms by means of real-time PCR

(76) Inventors: Ursula Obst, Karlsruhe (DE); Holger Volkmann, Karlsruhe (DE); Thomas Schwartz, Karlsruhe (DE); Silke Kirchen, Karlsruhe (DE)

Correspondence Address: Friedrich Kueffner, Suite 910, 317 Madison Avenue, New York, NY, 10017, US

(21) Appl. No.: 10/928,647

(22) Filed: Aug. 26, 2004

(30) Foreign Application Priority Data
Aug. 28, 2003 (DE) 103 39 609.8

(51) Int. Cl. C12Q 1/68; C07H 21/04; C12P 19/34

(52) U.S. Cl. 435/6; 435/91.2; 536/24.1

(57) ABSTRACT

An oligonucleotide, a method and a system for detecting antibiotic resistance-mediating genes in microorganisms by means of real-time PCR, comprising: the use of a first primer nucleotide sequence (A) which is selected from the group of sequences consisting of SEQ# 1-4, the use of a second primer nucleotide sequence (B) which is selected from the group of sequences consisting of SEQ# 5-8, with the sequences 1 and 5, 2 and 6, 3 and 7, and 4 and 8 being used as primer pairs, and the use of at least one first dye (C) for detecting the PCR-amplified DNA, and their use, in particular on a biochip.

The invention relates to novel oligonucleotides, and methods and systems, for detecting antibiotic resistance-mediating genes in microorganisms by means of real-time PCR using the novel oligonucleotides.

Antibiotics play an increasing role in regard to the influence of xenobiotics on the environment. Humans are increasingly introducing antibiotics into the environment by using them, too frequently and possibly incorrectly, as therapeutic agents or in feedstuffs, for the purpose of promoting growth in fattening cattle. In this connection, they can reach the environment, from anthropogenic sources, by way of a large number of entry routes and, in the environment, bring about an enrichment of antibiotic-resistant bacteria. Bacteria possessing multiple resistances, in particular, can then only be controlled with difficulty when they infect humans and animals. Even antibiotics which are highly active, and which are therefore nowadays used only as reserve antibiotics, i.e. to be employed when all the others have failed, can lose their effect as a result of resistance developing.

However, detecting antibiotics in the environment, for the purpose of determining the extent of the unnatural introduction, is frequently difficult and requires expensive analytical equipment. In addition to this, chemical analyses are unable to provide information with regard to the effect of the analysed substance on organisms.

However, the fact that antibiotics have an influence on bacterial populations can be demonstrated directly by an increase in resistant bacteria, with the spread of these bacteria in the environment representing a threat.

Normally, antibiotic-resistant bacteria are identified in culture experiments without the resistance-mediating genes being detected directly. Such classical microbial methods using antibiograms are tedious and restricted to detecting bacteria which can be cultured and do not permit any conclusions to be drawn with regard to the genetic causes of the resistances. Furthermore, relatively large quantities of the bacteria are required for this. Molecular biological methods which have been used thus far, such as conventional PCR assays, cannot be quantified.

The polymerase chain reaction (PCR) is a customary molecular biological method, which is known to the skilled person, for multiplying (amplifying), in a very short period of time, a few mol of any arbitrary genomic DNA sequence in vitro by factors of from 10⁶ to 10⁸ (cf. Rompp Lexikon Biotechnologie und Gentechnik [Römpp Encyclopedia of Biotechnology and Genetic Manipulation], 2nd edtn., Thieme Verlag Stuttgart 1999, “Polymerase chain reaction”, page 627).

Real-time PCR, which is derived from this, makes it possible to analyze (quantify) the amplification by detecting the fluorescence of a dye, with this fluorescence being directly or indirectly associated with the multiplication of the amplified DNA (cf. review in Journal of Molecular Endocrinology, 2000, Vol. 25, pp. 169-193).

George E. Killgore et al., Journal of Clinical Microbiology, July 2000, pages 2516-2519 “A 5′ Nuclease PCR (TaqMan) High-Throughput Assay for Detection of the mecA gene in Staphylococci” discloses that real-time PCR, using the TaqMan method (P. M. Holland et al. in Proc. Natl. Acad. Sci. USA 88: 7276-7280, 1991) should be used for rapidly investigating a large number of hospital patients for the presence of the mecA gene, which, in staphylococci, is responsible for resistance to the antibiotic methicillin.

However, the primers, and the probe for the mecA gene, which are used in that publication are not suitable for use on biochips since the primers employed are not capable of multiplexing, i.e. they are too long for achieving rapid and uniform kinetics. In addition, the article only discloses primers and a probe for the mecA gene. Other antibiotic resistance genes, and corresponding primers or probes, are not mentioned and are not used jointly, either.

However, in order for it to be possible to use primers, and, where appropriate, probes, for several genes simultaneously on biochips, these primers and probes must approximate to each other in regard to their kinetic properties, i.e. they must be capable of multiplexing. Otherwise, incorrect results would be obtained for a particular gene if the appurtenant primer pair, for example, had a more favorable kinetics than that of the other primer pairs.

The object of the present invention is to provide reliable and rapid systems for detecting and quantifying clinically relevant antibiotic-resistant bacteria in the environment by means of molecular biological detection systems which are transposable, in particular, to biochip technology and are furthermore species-specific.

Accordingly, the novel oligonucleotides comprising a nucleotide sequence selected from the group of sequences consisting of SEQ# 1-8 were found, with these oligonucleotides being suitable for use as primers for PCR, in particular real-time PCR.

Novel oligonucleotides comprising a nucleotide sequence selected from the group of sequences consisting of SEQ# 9-12 were also found, with these oligonucleotides being suitable for use as probes for the real-time PCR.

In addition, the method according to the invention for detecting antibiotic resistance-mediating genes in microorganisms by means of real-time PCR, with this method comprising:
the use of at least one first oligonucleotide (A) as claimed in claim 1 or 2 as primer, and
the use of at least one first dye (C) for detecting the PCR-amplified DNA,
   was found, with, in particular,
the first primer nucleotide sequence (A) being selected from the group of sequences consisting of SEQ# 1-4,
a second primer nucleotide sequence (B) being selected from the group of sequences consisting of SEQ# 5-8, and
the sequences SEQ# 1 and 5, 2 and 6, 3 and 7, and 4 and 8, being used as primer pairs.

The method according to the invention can be used to detect antibiotic resistance-mediating genes under real-time conditions and in a manner which is quantitatively species-specific and gene-specific. In other words, the primer nucleotide sequences were selected such that it is possible, when using them, to employ real-time PCR for carrying out antibiotic-specific and species-specific tests for detecting antibiotic resistance-mediating genes in microorganisms and total DNA from bacterial populations.

In particular, the length of the primer pairs of the antibiotic detection systems are aligned with each other in order to facilitate PCR in a multiplex assay; i.e. the method according to the invention and the primer nucleotide sequences which are employed therein, and also the probes which are described below, can be used to look for the presence of several antibiotic resistance-mediating genes simultaneously, that is in one “pot”.

The first primer nucleotide sequences (A), which are selected from the group of sequences consisting of SEQ# 1-5, are the forward primers. The second primer nucleotide sequences (B), which are selected from the group of sequences consisting of SEQ# 6-10, are correspondingly the reverse primers, with the sequences 1 and 6, 2 and 7, 3 and 8, 4 and 9, and 5 and 10 being employed as the primer pairs. The precise sequences are shown in FIG. 1.

The microorganisms of the genera Pseudomonas, Enterobacteriaceae, Staphylococcus and Enterococcus are particularly preferred for investigating the influence of man on his environment since they are found, in particular, in aqueous environmental samples. These microorganisms can be pathogenic facultatively. Of these microorganisms, particular preference is given to Pseudomonas aeruginosa, Enterobacter cloacae, Staphylococcus aureus and Enterococcus faecium.

Some of these microorganisms are used as bacteria for indicating fecal contamination or point to improper industrial regeneration processes, for example in drinking water technology.

With the increase in the frequency of bacterial resistance, the glycopeptide vancomycin plays an important role as a reserve antibiotic for treating infections with Gram-positive, resistant pathogens. However, vancomycin-resistant enterococci have already been detected in meat, chicken excrement, effluent water and even surface water. As the dominant resistance factor in enterococci, the vanA gene encodes a ligase which is able to alter the cell wall properties and in this way reduce the affinity for vancomycin.

Pseudomonas aeruginosa may be pathogenic and is frequently associated with nosocomial infections. In particular, species which harbor a bla_{VIM} gene exhibit resistance to β-lactamase-stable antibiotics such as imipenem. Apart from this clinical relevance, Pseudomonas aeruginosa is also present in the environment and has even been found in drinking water.

At present, seven variants of the imipenem resistance-mediating gene bla_{VIM} are known and have been sequenced. 18 imipenem-resistant Psendomonas aeruginosa strains were isolated from different resistance probe surfaces. Sequencing the bla_{VIM} gene showed that only the bla_{VIM}-2 gene was present. Primers and probe were therefore designed for specifically detecting bla_{VIM}-2.

The bla_{VIM} resistance genes are encoded on plasmids. In addition to being present in Pseudomonas aeruginosa, these genes were also found on the plasmids of other bacteria. The genes which are present on plasmids are subject to mechanisms of dissemination which are different from those to which genomically located genes are subject. Plasmid DNA can be exchanged between bacteria of the same and different species (horizontal gene transfer). For example, resistance genes which are coupled to other plasmid-bound genes can have an extremely positive effect on the survival of the bacterium while the lack of any selection pressure exerted by the antibiotic can have a negative effect on the persistence of the resistance gene in the cell. It is known that, when there is no selection pressure, bacteria are able to eliminate the corresponding plasmids from the cell. The bla_{VIM} gene can therefore serve as an indicator of the spread of resistance genes which are located on these mobile genetic elements.

The enterobacterial gene ampC is a frequently inducible, chromosomally encoded resistance gene for the synthesis of a β-lactamase which is able to hydrolyze penicillin G, ceftazidime and other broad-spectrum cephalosporins. Enterobacter cloacae harboring the ampc resistance gene are found in excrement and effluents.

Staphylococci are opportunistic bacteria and are frequently found in association with nosocomial infections. Almost 50 percent of all infections which occur in association with intensive care can be ascribed to Staphylococcus aureus or coagulase-negative staphylococci (CNS). Since the antibiotic methicillin began to be used, there has been a marked increase in the appearance of resistant Staphylococcus aureus and CNS which harbor the mecA gene, which is essential for methicillin resistance.

The antibiotics imipenem, ampicillin, methicillin and vancomycin, in particular, are therefore of interest because bacteria possessing resistances to these antibiotics are of clinical relevance and are good indicators of the contamination of aquatic systems with antibiotic-resistant bacteria.

For this reason, the antibiotic resistance-mediating genes from the group consisting of bla_{VIM}, ampc, mecA and vanA, which are responsible for resistance in the corresponding microorganisms, are likewise of particular interest and targets of the method according to the invention.

PCR-derived real-time PCR makes it possible to analyze (quantify) the amplification by detecting the fluorescence of a dye, which fluorescence is associated either directly or indirectly with the multiplication of the amplified DNA.

A direct method uses a dye which binds nonspecifically to double-stranded DNA and only fluoresces in connection with this binding. When the target DNA is amplified during the real-time PCR, this dye binds to the newly formed double-stranded DNA such that the measurable fluorescence increases.

Another direct method is that of using fluorescence resonance energy transfer (FRET) probes which bind to the amplified DNA. A FRET probe is a short oligo-nucleotide which is complementary to one of the strands of the target genome sequence. The probe comprises two fluorescent dyes, i.e. a “reporter” at the 5′ end, and a “quencher” at the 3′ end, of the probe. In the probes, the dyes are held, in the unbound state, in spatial proximity by means of a loop arrangement (hairpin loop). The hairpin loop is generated by means of complementary sequences which are present at the ends of the actual probe sequence. Because of its proximity to the reporter, the quencher dye is able to “quench”, i.e. extinguish, its fluorescence by means of the FRET. This probe, which is also termed a “beacon”, is used in the real-time PCR reaction together with the forward and reverse PCR primers. Binding of the probe to the PCR-amplified target DNA sequence which is complementary to the probe sequence disrupts the hairpin loop and thereby separates the two dyes, resulting in the FRET interference being abolished and the fluorescence of the reporter dye becoming measurable.

What is termed the “Taqman” method (C. A. Heid et al., Genom Res. 6, 986, 1996) is also a direct real-time PCR method. This method also employs a FRET probe which, in contrast to the abovementioned beacons, does not, however, possess any hairpin loop. While the polymerase enzyme is replicating the new DNA strand, the exonuclease activity degrades the FRET probe, which is bound to the target DNA, at its 5′ end such that the reporter dye is released from the probe. As a result, the reporter dye is no longer in the spatial vicinity of the quencher dye which means that its fluorescence is no longer quenched and can now be measured. The amplification of the target DNA, and, as a result, the increase in the release of the reporter dye, can then be detected using a suitable optical measuring system.

Another indirect method consists of a combination of the abovementioned beacons and the primers, with a primer being linked, via a nonamplifiable compound, to the beacon by way of its 5′ end. When the target DNA is amplified by the PCR, this probe, which is also termed a “scorpion”, becomes linked to the target DNA sequence, because of the primers, but is not itself amplified on account of the nonamplifiable compound. During the subsequent denaturation step, the probe sequence which is complementary to the target DNA can bind, as a result of the hairpin loop being disrupted, to the target DNA sequence in connection with the following cooling. As a result of the hairpin loop being disrupted, the two dyes are prevented from being in spatial proximity and the fluorescence of the reporter dye can be measured (cf. above). A modification of these “scorpions” consists in the dyes being separated into two different oligonucleotides, resulting in the signal intensity being improved. Thus, the loop configuration is replaced by two complementary strands, with the quencher dye no longer being arranged at the 3′ end of the probe sequence but instead being arranged at the 3′ end of its own strand, with this 3′ end facing the 5′ end of the probe. Consequently, the dyes are only in spatial proximity when the complementary strands are bound. The denaturation and subsequent cooling results in the actual probe sequence being separated from the quencher-carrier sequence and thereby permits the abovementioned binding of the probe to the target DNA and the detection of the reporter dye fluorescence.

For further clarification, the reader is referred to Science, Vol. 296, pages 557-558, Apr. 19, 2002, and Journal of Molecular Endocrinology 2002, 29, pages 23-29, and www.dxsgenotyping.com.

The primers according to the invention are consequently used for detecting an antibiotic-specific nucleotide sequence from the genes to be detected. The dyes, or the probes and dyes, in turn label the amplified antibiotic resistance-mediating gene sequences to enable them to be detected by means of fluorescence measurements. The course of the amplification is used to establish a threshold value at which the fluorescence signal of the reporter dye is clearly greater than the background signal and the amplification of the target DNA is proceeding under nonlimiting conditions (linear range). A given value, which is a measure of the quantity of target DNA employed, is obtained from the intersection of the threshold value line and the amplification curve. Standards containing known initial quantities permit calibration, which then makes it possible to determine the absolute value for the quantity of target DNA, i.e. of the resistance gene.

The primer/probe systems according to the invention are selected such that they can be immobilized on support materials. These are customarily gold, glass, silicon compounds, etc., which are known to the skilled person.

Very particularly, the primer/probe systems according to the invention are suitable for being used on biochips. Thus, the primers and probes can be applied to, and immobilized on, supports using nanospotters, for example.

The first dye (C) for detecting the PCR-amplified DNA is consequently either a direct DNA dye or a reporter dye, which is then used together with the second dye (quencher).

It is therefore advantageous if the at least one first dye (C) fluoresces on binding to the DNA double strand. It is then possible to implement the above-mentioned first direct real-time PCR method. The dyes are commercially available dyes which are known to the skilled person and which fluoresce on intercollation in the DNA or RNA double strand. This thereby makes it possible to detect the double strands which are newly formed during the PCR. The dye SYBR Green is particularly frequently employed.

If a probe nucleotide sequence (D) selected from the group of sequences consisting of SEQ# 9-12 is also employed, it is then possible to use the primers according to the invention to carry out one of the other direct or indirect real-time PCR methods. The sequences of the probes are also given in FIG. 1.

For this, it is advantageous if the at least one first dye (C) is linked to the probe nucleotide sequence (D), in particular by way of its 5′ end.

If one of the primer nucleotide sequences (A, B) is linked to the probe nucleotide sequence (D) by way of its 3′ end and the 3′ end is furthermore linked to the primer nucleotide sequence (A, B) by way of a compound (E) which cannot be amplified by PCR, the method which is used can then be the particularly promising indirect method of “scorpions”, which is distinguished, in particular, by its rapid unimolecular reaction.

A second dye (F) is also required, which dye can be linked directly to the probe nucleotide sequence (D) and, when spatially proximal, extinguishes the fluorescence of the first dye (C) by means of what is termed FRET (fluorescence resonance energy transfer).

This dye can advantageously be linked to the probe nucleotide sequence (D) by way of its 3′ end. This thereby results in a unipartite “scorpion”. The probe nucleotide sequence (D) is then held in a hairpin loop configuration by means of complementary sequences at its 5′ and 3′ ends. Consequently, the first dye (reporter) and the second dye (quencher) are located close to each other spatially and no fluorescence occurs (cf. above).

Alternatively, it is possible for the at least one second dye (F) to be linked to a sequence (G) which is complementary to the probe nucleotide sequence (D) by way of the 3′ end of the (G) sequence. This then results in a bipartite “scorpion” (cf. above).

If, when the probe nucleotide sequence (D) has a hairpin loop configuration, the link to the primer nucleotide sequence is dispensed with and the primers are added individually in the normal manner, this then results in what is termed a “beacon”, which likewise only fluoresces in the bound state (cf. above).

The invention furthermore encompasses a system for use in the above-described method for detecting antibiotic resistance-mediating genes in microorganisms by means of real-time PCR, with the system comprising:
a first primer nucleotide sequence (A) which is selected from the group of sequences consisting of SEQ# 1-4,
a second primer nucleotide sequence (B) which is selected from the group of sequences consisting of SEQ# 5-8, with sequences 1 and 5, 2 and 6, 3 and 7, and 4 and 8 being used as primer pairs, and
at least one first dye (C) for detecting the PCR-amplified DNA,
where appropriate, a probe nucleotide sequence (D) which is selected from the group of sequences consisting of SEQ# 9-12,
where appropriate, a second dye (F) which, when spatial proximity, extinguishes the fluorescence of the first dye (C).

The invention is described below using examples.

Reference Bacterial Strains

Enterococcus faecium B7641 vanA^{r} was used as the reference strain for the vanA gene. The strains Staphylococcus aureus AlmecA^{r} and Enterobacter cloacae A10ampCᵣ were identified taxonomically both by sequencing and by way of their resistance genes and were in each case used as references. Pseudomonas aeruginosa 15 was isolated and likewise identified taxonomically using the API 20NE kit (bioMerieux, Nürtingen, Germany) and employed as the reference for bla_{VIM}-2. The strain Pseudomonas aeruginosa VR 143/97 was used as the reference for the bla_{VIM}-1 gene.

The antibiotic-sensitive control strains employed were Staphylococcus aureus ssp. aureus DSM 20231 mecAS, Enterococcus faecium DSM 20477 vanAs, Escherichia coli DSM 1103 ampcs, for sensitive Enterobacteriaceae, and Pseudomonas aeruginosa 22 VIM^{S}.

Sampling and Preparation

Water samples (500 ml) were withdrawn, for culturing and DNA extraction, from the influent water, sewage sludge and effluent of public sewage disposal plants and from the effluent from hospitals (clinical effluent).

Enterococci were enriched by culturing them at 37° C. for 24 h in azide-dextrose broth (Oxoid, Basingstoke, England). Vancomycin-resistant isolates were obtained by means of selection on kanamycin-esculin-azide agar (Merck KG aA, Darmstadt, Germany) containing 32 μg of vancomycin per ml, in accordance with NCCLS. Because of the high incidence of Enterobacteriaceae in effluent, isolates were obtained by culturing on Chromocult agar (Merck KG aA, Darmstadt, Germany) containing 32 μg of ceftazidime/ml, as the antibiotic for the resistance selection, without any prior enrichment.

Reference strains of Enterobacter cloacae and Enterococcus faecium were suspended, and diluted in a decreasing series in PBS (137 mM NaCl, 7.25 mM Na₂HPO4, 0.2 mM KH₂PO4, 2.7 mM KCL, pH 7.4) and cultured, for quantification by means of plate count, on R2A agar (Difco) or Slanetz-Bartley agar (Merck).

Primer/Probe Design

The sequences of the resistance genes were taken from the NCBI database:

| Gene | Number |
|---|---|
| Enterobacter cloacae ampC | AF411145 |
| Pseudomonas aeruginosa bla_{VIM} | Y18050 |
| Staphylococcus aureus mecA | E09771 |
| Enterococcus faecium plasmid pIP816 vanA | X56895 |

The Applied Biosystems Primer Express software was employed to develop the primer and probe sequences for use in a standardized TaqMan amplification protocol. All the primers and fluorogenic probes were synthesized by the company Applera (Darmstadt, Germany).

The specificity of the primers and probes was established by using BLAST methods to compare their sequences with the NCBI entries. The corresponding antibiotic-sensitive control strains were tested for a crossreaction. In addition, the primer/probe systems according to the invention were tested by means of PCR which was carried out three times using serially diluted reference strains and Ct calibration lines were established (FIG. 2).

PCR

The company Applied Biosystems uses a universal Master Mix (uMM) which is optimized for preparing quantitative PCR assays and which contains dNTPs, AmpliTaq Gold® DNA polymerase, AmpErase® UNG (uracil-N-glycosidase), MgCl₂ and buffer components, and also the fluorogenic dye ROX as passive reference, such that the analytical software is able to correct pipetting errors automatically.

The AmpliTaq Gold (Applied Biosystems) polymerase, which is used in the TaqMan PCR, is a recombinant form of the AmpliTaq DNA polymerase and was initially activated irreversibly by means of a 9 to 12-minute incubation step at 90° C.

In order to optimize the probe hybridization, a two-step PCR was carried out under standard conditions. This is made possible by the significant activity of the AmpliTaq Gold polymerase at temperatures of >55° C. and a selection of primers having a uniform annealing temperature of about 60° C. This makes it possible to carry out a standardized two-step PCR protocol in which the amplification only requires a 95° C. step, for the denaturation, and a 60° C. step, for the annealing and extension.

In order to protect against contamination, a two-minute incubation step with the AmpErase UNG was first of all carried out at 50° C.

An ABI 7000 or 7700 sequence detector system (Applied Biosystems) was used for the real-time PCR amplification.

For carrying out the PCR, 10 μl of a template (sample to be analyzed), which were amplified in 50 μl reaction volumes which contained 300 nM of each primer, 200 nM of a FAM/TAMARA-labeled probe and 25 μl of 2-fold TaqMan universal Master Mix and 7 μl of water, were subjected to a standard TaqMan temperature profile (2 min at 50° C., 10 min at 95° C. and 40 cycles of in each case 15 s at 95° C. and 1 min at 60° C.).

Taxonomic and Resistance-Gene Identification by Means of Sequencing

Strains were identified taxonomically by partially sequencing the 16S rDNA. The universal primers 27F (5′-AGAGTTTGATCMTGGCTCAG-3′, SEQ ID 13) and 517R (5′-ATTACCGCGGCTGCTGG-3′, SEQ ID 14) (Muyzer et al., Appl. Enrivon. Microbiol. 59(3), 695, 1993; Kilb et al., Acta Hydrochim. Hydrobiol. 26(6), 349, 1998) were used for generating a 526 base pair amplicon of sites 8 to 534 of the E. coli 16S rDNA (Brosius et al., J. Mol. Biol. 147, 107, 1981). A PCR profile having 35 cycles consisting of 94° C. for 30 s, 49° C. for 30 s and 72° C. for 1 min, after activating the HotStart Taq polymerase (Qiagen, Hilden) at 95° C. for 15 min, and a final extension cycle at 72° C. for 7 min, were used.

The 27F primer was also employed for the sequencing reaction. In order to test for the presence of the resistance gene vanA, a given PCR product was amplified using the primers vanA1 (5′-TCTGCAATAGAGATAGCCGC-3′, SEQ ID 15) and vanA2 (5′-GGAGTAGCTATCCCAGCATT-3′, SEQ ID 16) (Klein et al., Appl. Environ. Microbiol. 64, 1825, 1998). The primer vanA1 was then used as the primer for the sequencing. The ampC resistance gene was amplified in accordance with Schwartz et al. (FEMS Microbiol. Ecoli. 43(3), 325, 2003) using the primers ampC-For (5′-TTCTATCAAMACTGGCARCC-3′, SEQ ID 17) and ampC-Rev (5′-CCYGTTTTATGTACCCAYGA-3′, SEQ ID 18). The resistance gene mecA was amplified in accordance with Murakami et al. J. Clin. Microbiol. 29, 2240, 1991) using the primers mecA1 (5′-AAAATCGATGGTAAAGGTTGGC-3′, SEQ ID 19) and mecA2 (5′-AGTTCTGCAGTACCGGATTTGC-3′, SEQ ID 20). All the amplifications were carried out using an Applied Biosystems GeneAmp PCR System 9700.

The PCR products were sequenced by the Sanger method (Sambrook et al., Molecular cloning: a laboratory manual, Harbor Laboratory Press, Cold Spring Harbor, N.Y. 2001) using the Applied Biosystems BigDye Terminator Cycle Sequencing Ready Reaction Chemistry Kit. The sequencing reaction was begun with a denaturation step at 95° C. for 5 min, with this being followed by 25 cycles at 55° C. for in each case 5 s and terminated with an extension reaction at 60° C. for 1 min (Applied Biosystems GeneAmp PCR System 9700). The fragments which were obtained were separated and analyzed using an Applied Biosystems ABI Prism 310 genetic analyzer. The resulting DNA sequences were used to carry out BLAST DNA homology searches in the NCBI database.

Results

The effluents from five public sewage disposal plants were examined for the presence of antibiotic-resistant bacteria. Vancomycin-resistant enterococci and P-lactam-resistant Enterobacteriaceae were isolated from all the effluent samples following specific enrichment.

The isolates were first of all identified biochemically as Enterococcus faecium and Enterobacter cloacae using the rapid ID32 strep and API 20E test kits (bioMerieux, Nurtingen, Germany). These results were confirmed by carrying out sequence analyses based on the 16S rDNA. The enterococcal strains from EF1 to EF4 exhibited 99 to 100% homology with Enterococcus faecium and the isolated Enterobacteriaceae EB4, EB86, EB101 and EB102 exhibited 98 to 100% homology with Enterobacter cloacae.

Previous investigations (Schwartz et al., cf. above) and culturing experiments had shown that it was not possible to isolate any staphylococci from samples of public effluents. For this reason, clinical isolates S1 to S4, which were methicillin-resistant staphylococci obtained from patients at Heidelberg University, were used for the real-time PCR experiments. Their taxonomic identity as Staphylococcus aureus was confirmed by a 99% homology with the NCBI database entries.

The above-described resistant bacteria were used to carry out specific PCR experiments for amplifying the resistance genes vanA, ampC and mecA. Both the PCR results and the subsequent sequencing showed that the resistance of the enterococci was elicited by the vanA gene, while the resistance of the Enterobacter cloacae and E. coli was elicited by the ampc gene and the methicillin resistance of the staphylococci was elicited by the mecA resistance gene. All the resistant strains exhibited a homology of 99 to 100% with the NCBI database entries.

The primer/probe systems which were developed are shown in table 1.

The Cₜ values (table 2) were ascertained by amplifying and plotting the results from samples of serially diluted DNA from the reference strains. FIG. 1 shows this, by way of example, for Enterobacter cloacae ampC.

Subsequently, these data were used for generating the straight calibration lines which are shown in FIG. 2. In a semilogarithmic plot, the linear data regions represent the measurement regions of the primer/probe systems which are quantifiable.

In order to avoid falsely positive results, control experiments without template (NTC, no template control), i.e. without bacterial DNA, and without complementary sequence (NAC, no amplification control) in the bacterial DNA were performed in all the PCR assays (cf. table 2).

35 effluent samples from five sewage disposal plants, and two hospital effluent samples, were examined for the occurrence of the resistance genes vanA, ampc and mecA. Suitable commercially available extraction kits were used to extract between 9 and 100 μg of total DNA from 30 to 50 ml sample volumes. This DNA was used for the abovementioned TaqMan systems in the real-time PCR. ampc was found in 78% of the samples. The apertinent Ct values are listed in table 1. At 22%, the vanA gene occurred much less frequently while the mecA gene did not occur in detectible concentration in the effluents.

However, it was possible to use culturing methods to isolate Staphyloccus aureus, as well as resistant Enterococcus faecium and Enterobacter cloacae, from the corresponding environmental habitats. Table 3 also shows that the corresponding resistance genes were detected in some of these isolates; the table also includes the apertinent Cₜ values.

Carrying out Taqman PCR on the reference strains showed that the vim1 Taqman system enables the bla_{VIM}-2 gene to be detected whereas bla_{VIM}-1 is not detected (table 4).

For investigations into the occurrence of the bla_{VIM} gene, effluent samples containing different clinical contents were examined. In this connection, it was possible, following DNA extraction, to detect the presence of bla_{VIM}-2 in an effluent sample without any prior enrichment of the target organisms (cf. table 4).

TABLE 1

| | | | | | |
|---|---|---|---|---|---|
| Resistance | | Target | TaqMan | | |
| gene | Antibiotic | organism | system | Primer sequences | Probe sequence |

| | | | | | |
|---|---|---|---|---|---|
| bla_{VIM} | imipenem | Pseudamonas | vim1 | vim1FP: 5′- | vim1: 5′- |
| | | aeruginosa | | cctccattgag- | caacactacccgga- |
| | | | | cggattca-3′ | agcacagttcgtc-3′ |
| | | | | (SEQ #1) | (SEQ#9) |
| | | | | vim1RP 5′- | |
| | | | | gccgtgccccg- | |
| | | | | gaa-3′ | |
| | | | | (SEQ #5) | |
| ampC | ampicillin | Enterobacter | ampC | Lak1FP: 5′- | P-Lak1: ′5′- |
| | | cloacae | | gggaatgctgga- | cctatggcgtgaaa- |
| | | | | tgcacaa-3′ | accaacgtgca-3′ |
| | | | | (SEQ #2) | (SEQ #10) |
| | | | | LakA1RP: 5′- | |
| | | | | catgacccagtt- | |
| | | | | cgccatatc-3′ | |
| | | | | (SEQ #6) | |
| mecA | methicilin | CNS, | mecA1 | mecA1 FP 5′- | mecA1: 5′- |
| | | Staphylo- | | cgcaacgttcaa- | aatgacgctatgat- |
| | | coccus aureus | | tttaattttgtt | cccaatctaacttc- |
| | | (MRSA) | | aa-3′ | caca-3 |
| | | | | (SEQ #3) | (SEQ #11) |
| | | | | mecA1 RP: 5′- | |
| | | | | tggtctttctgc- | |
| | | | | attcctgga-3′ | |
| | | | | (SEQ #7) | |
| vanA | vancomycin | Entero- | vana3 | vanA3FP: 5′- | vanA3: 5′- |
| | | coccus | | ctgtgaggtcgg- | caactaacgcg- |
| | | faecium | | ttgtgcg-3′ | gcactgtttcc- |
| | | | | (SEQ #4) | caat-3′ |
| | | | | | (SEQ #12) |
| | | | | vanA3RP: 5′- | |
| | | | | tttggtccacdc- | |
| | | | | gcca-3′ | |
| | | | | (SEQ #8) | |

TABLE 2

| | | | | Ct of sensitive | |
|---|---|---|---|---|---|
| TaqMan | Threshold value | | | reference bacteria | |
| system | (ΔRn) | Ctₘᵢₙ | Ctₘₐₓ | (NAC) | Ct_{NTC} |
| vim1 | 0.22 | 20.7 | 31.1 | >40 | >40 |
| ampC | 0.19 | 20.6 | 38.0 | >40 | >40 |
| mecA1 | 0.20 | 22.1 | 39.3 | >40 | >40 |
| vana3 | 0.29 | 22.8 | 38.2 | >40 | >40 |

NTC: no template control
NAC: no amplification corntrol

TABLE 3

| | Type of sample | Sample | Cₜ VALUE |
|---|---|---|---|
| vanA | Reference | Enterococcus faecium | 16.0 |
| | | B7641 | |
| | Sensitive reference | Enterococcus faecium DSM | >40 |
| | | 20477 | |
| | Resistant isolates | EF 1 | 15.6 |
| | | EF 2 | 16.0 |
| | | EF 3 | 18.0 |
| | | EF 4 | 16.5 |
| | Total DNA, effluent | ww2 | 31.9 |
| | | ww3 | 34.7 |
| | | ww6 | 33.7 |
| | | ww7 | 27.6 |
| | | 15 samples | >40 |
| ampC | Reference | Enterobacter cloacae P | 15.4 |
| | | A10 | |
| | Sensitive reference | E. coli DSM 1103 | >40 |
| | Resistant isolates | EB 4 | 19.4 |
| | | EB 86 | 18.2 |
| | | EB 101 | 18.4 |
| | | EB 102 | 18.9 |
| | Total DNA, effluent | ww8 | >40 |
| | | ww9 | >40 |
| | | ww10 | 34.5 |
| | | ww11 | 35.3 |
| | | ww12 | 27.3 |
| | | ww13 | 29.1 |
| | | ww14 | 28.1 |
| | | ww15 | 31.4 |
| | | ww16 | 29.4 |
| mecA | Reference | S. aureus A1 | 20.5 |
| | Sensitive reference | S. aureus DSM 20231 | >40 |
| | Resistant isolates | S1 | 21.3 |
| | | S2 | 19.7 |
| | | S3 | 19.7 |
| | | S4 | 19.4 |
| | Total DNA, effluent | ww8 | >40 |
| | | ww9 | 38.6 |
| | | ww17 | >40 |
| | | ww18 | >40 |
| | | ww19 | >40 |
| | | ww20 | >40 |
| | | ww21 | 38.6 |

TABLE 4

| | Type of sample | Sample | Cₜ VALUE |
|---|---|---|---|
| bla_{VIM} | Reference bla_{VIM}-2 | Ps. aeruginosa 15 | 20.7 |
| | Reference bla_{VIM}-1 | Ps. aeruginosa VR | >40 |
| | | 143/97VR | |
| | Sensitive reference | Ps. aeruginosa 22 | >40 |
| | Resistant effluent isolates | Ps. aerug. 1 | 22.2 |
| | | Ps. aerug. 9 | 22.7 |
| | | Ps. aerug. 15 | 23.1 |
| | | Ps. aerug. 16 | 22.4 |
| | | Ps. aerug. 23 | 23.0 |
| | | Ps. aerug. 39 | 18.5 |
| | | Ps. aerug. 49 | 19.3 |
| | | Ps. aerug. 56 | 17.1 |
| | | Ps. aerug. 72 | 17.9 |
| | | Ps. aerug. 76 | 17.3 |
| | Resistant influent | Ps. aerug. 81 | 20.8 |
| | water isolates | Ps. aerug. 83 | 18.3 |
| | Total DNA, effluent | wwB1 | >40 |
| | | wwB2 | >40 |
| | | wwB3 | 35.4 |
| | | wwB4 | >40 |
| | | wwB5 | >40 |

[image]

[image]

1. An oligonucleotide which comprises a nucleotide sequence which is selected from the group of sequences consisting of seq# 1-8.

2. An oligonucleotide as claimed in claim 1, which can be used as a primer for PCR.

3. An oligonucleotide which comprises a nucleotide sequence which is selected from the group of sequences consisting of SEQ# 9-12.

4. An oligonucleotide as claimed in claim 2, which can be used as a probe for real-time PCR.

5. A method for detecting antibiotic resistance-mediating genes in microorganisms by means of real-time PCR, which comprises:
the use of at least one first oligonucleotide (A) as claimed in claim 1 as primer, and
the use of at least one first dye (C) for detecting the PCR-amplified DNA.

6. The method as claimed in claim 5, wherein
the first primer nucleotide sequence (A) is selected from the group of sequences consisting of SEQ# 1-4,
a second primer nucleotide sequence (B) is selected from the group of sequences consisting of SEQ# 5-8, and
the sequences SEQ# 1 and 5, 2 and 6, 3 and 7, and 4 and 8, are used as primer pairs.

7. The method as claimed in claim 5, wherein the at least one first dye (C) fluoresces on binding to the DNA double strand.

8. The method as claimed in claim 5, wherein an oligonucleotide which comprises a nucleotide sequence which is selected from the group of sequences consisting of SEQ# 9-12 is used as probe.

9. The method as claimed in claim 8, wherein the at least one first dye (C) is linked to the probe nucleotide sequence (D), in particular by way of its 5′ end.

10. The method as claimed in claim 8, wherein one of the primer nucleotide sequences (A, B) is linked to the probe nucleotide sequence (D) by way of its 3′ end.

11. The method as claimed in claim 8, wherein the 3′ end of the probe nucleotide sequence (D) is linked to a primer nucleotide sequence (A, B) by way of a compound (E) which cannot be amplified by PCR.

12. The method as claimed in claim 5, wherein a second dye (F) is linked to the probe nucleotide sequence (D), which dye, when spatially proximal, extinguishes the fluorescence of the first dye (C).

13. The method as claimed in claim 12, wherein the at least one second dye (F) is linked to the probe nucleotide sequence (D) by way of its 3′ end.

14. The method as claimed in claim 8, wherein the probe nucleotide sequence (D) is held in a hairpin loop configuration by means of complementary sequences at its 5′ and 3′ ends.

15. The method as claimed in claim 8, wherein the at least one second dye (F) is linked to a sequence (G) which is complementary to the probe nucleotide sequence (D) by way of the 3′ end of the (G) sequence.

16. The method as claimed in claim 5, wherein the antibiotics are selected from the group consisting of imipinem, ampillin, methicillin and vancomycin.

17. The method as claimed in claim 5, wherein the antibiotic resistance-mediating genes are selected from the group consisting of blavim, ampc, mecA and vanA.

18. The method as claimed in claim 5, wherein the microorganisms are selected from the group consisting of Pseudomonas aeruginosa, Enterobacter cloacae, Staphylococcus aureus and Enterococcus faecium.

19. The method as claimed in claim 5, wherein the nucleotide sequences are immobilized on a support material, in particular on a biochip.

20. A system for detecting antibiotic resistance-mediating genes in microorganisms which comprises:
a first primer nucleotide sequence (A) which is selected from the group of sequences consisting of SEQ# 1-4,
a second primer nucleotide sequence (B) which is selected from the group of sequences consisting of SEQ# 5-8, with the sequences 1 and 5, 2 and 6, 3 and 7, and 4 and 8 being used as primer pairs, and
at least one first dye (C) for detecting the PCR-amplified DNA,
where appropriate, a probe nucleotide sequence (D) which is selected from the group of sequences consisting of SEQ# 9-12,
where appropriate, a second dye (F) which, when spatially proximal, extinguishes the fluorescence of the first dye (C).

21. The use of a system as claimed in claim 20, for immobilization on a support material, in particular on a biochip.

22. The use of a support material, in particular a biochip, which is provided with a system as claimed in claim 20, for detecting antibiotic resistance-mediating genes in micro-organisms by means of PCR.
